(19) United States
(12) Patent Application Publication
(10) Pub. No.: US 2025/0127385 A1
(43) Pub. Date: Apr. 24, 2025

(54) MEDICAL DEVICE SHAFTS AND RELATED METHODS OF MANUFACTURE

(71) Applicant: Boston Scientific Scimed, Inc., Maple Grove, MN (US)

(72) Inventors: Gene Thomas STORBECK, Millis, MA (US); Nestor Allan IBANEZ, Brighton, MA (US); Kyle Steven MEYERS, Franklin, MA (US); Pat S PHONGSAVANH, Blackstone, MA (US); Vincent P. LILIENTHAL, Plymouth, MN (US); Kyle Logan LEMKE, Hudson, WI (US); Matthew HART, Maple Grove, MN (US); Jonathan NEWINSKI, Minneapolis, MN (US)

(73) Assignee: Boston Scientific Scimed, Inc., Maple Grove, MN (US)

(21) Appl. No.: 18/920,145

(22) Filed: Oct. 18, 2024

Related U.S. Application Data
(60) Provisional application No. 63/591,809, filed on Oct. 20, 2023.

(51) Int. Cl. A61B 1/018; A61B 1/00; A61B 1/005; A61B 1/015

(52) U.S. Cl. CPC A61B 1/018; A61B 1/0011; A61B 1/00137; A61B 1/0052; A61B 1/0057; A61B 1/015

(57) ABSTRACT

A medical device shaft may include a tubular member, a braided tube, and an outer tube. The tubular member may define a working channel capable of receiving an accessory instrument. The braided tube may surround and contact the tubular liner. The outer tube may surrounding and contact the braided tube.

### CROSS-REFERENCE TO RELATED APPLICATIONS

This application claims the benefit of priority to U.S. Provisional Application No. 63/591,809, filed on Oct. 20, 2023, which is incorporated by reference herein in its entirety.

### TECHNICAL FIELD

This disclosure generally relates to medical devices, and particularly shafts for use in medical devices, and related methods of manufacturing such as shafts.

### BACKGROUND

Medical devices are often inserted into the body to perform a therapeutic and/or diagnostic procedure inside a subject's body. An example of such a device is an endoscope or other type of scope or insertion device, which includes an insertion portion that is introduced into the body for diagnostic or therapeutic purposes. An insertion portion of an endoscope is inserted into the subject's body through an opening (e.g., a natural opening or an incision) and is delivered to a site inside the body, for example, through a body lumen. In one example, an endoscope may be inserted into a subject's mouth and advanced through the subject's esophagus.

The insertion portion of an endoscope or other scopes and like devices includes a long shaft that extends from outside the body to the treatment site. The shaft should have a desired flexibility to traverse tortuous anatomy and typically has one or more lumens therethrough to achieve the desired functionality (e.g., irrigation, articulation, suction, accessory device delivery, etc.).

### SUMMARY

Examples of this disclosure relate to, among other things, medical devices having long, flexible shafts capable of achieving various diagnostic and therapeutic functions, and related methods of manufacturing such shafts.

According to one aspect, a medical device shaft may include a tubular member, a braided tube, and an outer tube. The tubular member may define a working channel capable of receiving an accessory instrument. The braided tube may surround and contact the tubular liner. The outer tube may surrounding and contact the braided tube.

In aspects, the tubular liner may include an inner layer and an outer layer surrounding the inner layer. The inner layer may be comprised of a polyethylene. The outer layer may be comprised of an acid-modified polyethylene. The inner layer may be co-extruded with the outer layer. The polyethylene of the inner layer may have a molecular weight of at least 200 kilodaltons. The outer layer may adhere the braided tube to the inner layer.

In aspects, the tubular liner may include only one layer. The only one layer may be comprised of an inner layer. The inner layer may be comprised of a polyethylene having a molecular weight of at least 200 kilodaltons.

In aspects, the braided tube may surround only one lumen. The only one lumen may comprise the working channel. The braided tube may include metal wires, and adjacent windings of the braided tube may contact one another.

In some aspects, the outer tube may be an extruded thermoplastic defining multiple lumens. The multiple lumens may include at least one steering lumen for accommodating a steering wire, at least one irrigation lumen capable of providing irrigation fluid, and at least one electronics lumen for accommodating electrical circuitry. Each of the at least one steering lumen, the at least one irrigation lumen, and the at least one electronics lumen may be surrounded by only the outer tube.

In aspects, the medical device shaft may consist essentially of the tubular liner, the braided tube, and the outer tube. The tubular liner may be extruded on a mandrel to form the working channel. The braided tube may be formed onto the tubular liner while the tubular liner is on the mandrel. The outer tube may be extruded onto the braided tube while the tubular liner is on the mandrel.

In aspects, the medical device may further comprise a handle directly attached to a proximal end of the medical device shaft and an end cap directly attached to a distal end of the medical device shaft. The handle may include steering components, and a port in communication with the working channel. The end cap may include an imager, lighting, and an exit port in communication with the working channel.

According to another aspect, a medical device shaft may comprise a tubular liner, a braided tube of metal wires, and a first thermoplastic outer tube. The tubular liner may include a polyethylene layer and a tie layer surrounding the polyethylene layer. The polyethylene layer may define a working channel capable of receiving an accessory instrument. The braided tube may surround contact the tie layer. The tie layer may enhance adhesion of the braided tube to the polyethylene layer. The braided tube may surround only one lumen comprising the working channel. The first thermoplastic outer tube may surround and contact the braided tube. The first outer tube may define a first plurality of lumens therethrough. The first plurality of lumens may be surrounded by only the first outer tube.

In aspects, the medical device shaft may further comprise a second thermoplastic outer tube surrounding and contacting the braided tube. The second outer tube may be distal to the first outer tube. The second outer tube may define a second plurality of lumens therethrough. The second plurality of lumens may be surrounded by only the second outer tube. The second outer tube may have a stiffness that is less than a stiffness of the first outer tube.

Aspect of this disclosure include methods of making a medical device shaft. The method may include extruding a tubular liner onto a mandrel; forming a braided tube onto the tubular member is on the mandrel; and, after the forming step, extruding an outer tube onto the braided tube while the tubular liner is on the mandrel.

In aspects, extruding the tubular liner onto the mandrel may include co-extruding an inner polyethylene layer and a tie layer surrounding the inner polyethylene layer.

In aspects, the outer tube may include a thermoplastic. The outer tube may define multiple lumens surrounded by only the outer tube. The multiple lumens may include at least one steering lumen for accommodating a steering wire, at least one irrigation lumen capable of providing irrigation fluid, and at least one electronics lumen for accommodating electrical circuitry.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of a medical system, according to aspects of this disclosure.

FIG. 2 is a cross-sectional view of a shaft of the medical system of FIG. 1, according to aspects of this disclosure.

### DETAILED DESCRIPTION

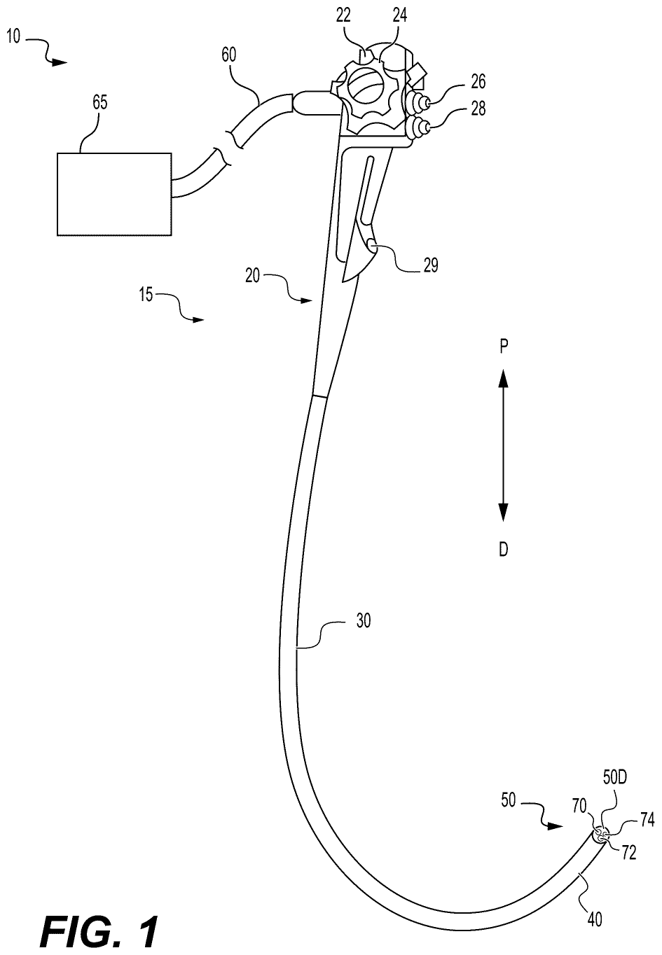

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when introducing the device into the subject. Proximal and distal directions are labeled with arrows marked “P” and “D,” respectively, in FIG. 1.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. Additionally, terms that indicate the geometric shape of a component/surface encompass both exact and approximate shapes.

Although a target site is discussed herein as being in the subject's gastrointestinal tract, this disclosure is not so limited, as the target site may be any internal lumen, organ, cavity, or other tissue within the subject. Additionally, although endoscopes are referenced herein, it will be appreciated that the disclosure encompasses various devices that may be inserted into a body of a subject, such as ureteroscopes, cholangioscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

This disclosure is described with reference to exemplary medical devices, assemblies, and/or systems having a flexible shaft for insertion in a body lumen. It should be noted that reference to any particular device and/or any particular procedure is provided only for convenience and not intended to limit the disclosure. A person of ordinary skill in the art would recognize that the concepts underlying the disclosed devices and application methods may be utilized in any suitable procedure, medical or otherwise. The assemblies and systems described herein may be used in conjunction with other types of medical devices. This disclosure may be understood with reference to the following description and the appended drawings, wherein like elements are referred to with the same reference numerals.

Referring to FIG. 1, a medical system 10 according to an exemplary embodiment is shown. Medical system 10 may include a medical device 15 comprising a handle 20 and an insertion portion 30 (including, e.g., a shaft). Insertion portion 30 may be connected to and extend from a distal portion of handle 20. Insertion portion 30 may terminate distally in a distal tip 50. In embodiments, the distal end of medical device 15 may include an end cap (not shown) that incorporates components at the distal end. In such a case, insertion portion 30, and particularly the shaft, may terminate at the proximal end of the end cap.

A distal face 50D of distal tip 50 may include one or more openings 70 (e.g., distal openings of working channels or lumens) for suction, irrigation, insufflation, accessory devices, etc. Moreover, in some aspects, distal face 50D includes one or more imaging devices 72 and/or one or more illumination devices (e.g., LEDs) 74. Imaging device(s) 72 may include one or more cameras, image sensors, lenses, etc. Additionally or alternatively, imaging device(s) 72 and/or LEDs 74 may be disposed on a side surface of a distal end portion 40 of insertion portion 30, for example, at or adjacent to distal tip 50. In further examples, imaging device(s) 72 and/or LEDs 74 may be disposed anywhere along a length of insertion portion 30 and/or distal end portion 40. In some examples, distal tip 50 may further include one or more end effectors (e.g., graspers, electrodes, biopsy devices, etc.).

An umbilicus 60 may extend from a proximal portion of handle 20. Umbilicus 60 may be removably coupled (e.g., directly or indirectly) to a processing unit 65. Processing unit 65 may be configured to process information (e.g., sensor data, imaging data, light data, etc.) received from medical device 15. In some aspects, processing unit 65 may be a controller associated with medical device 15. Imaging device(s) 72 and/or LEDs 74 may be electrically coupled (e.g., directly or indirectly) to processing unit 65, for example, via one or more wires and/or cables extending through insertion portion 30, through handle 20, and through umbilicus 60. For example, umbilicus 60 may include one or more electrical cables and/or light cables for coupling to processing unit 65 via, e.g., a removable connector.

Although not shown, processing unit 65 may include a visual output (e.g., an embedded monitor or screen), or processing unit 65 may be coupled to a visual output (e.g., an external or separate monitor or screen coupled to processing unit 65). Although not shown, umbilicus 60 may additionally or alternatively include one or more lumens for supplying a gas or a liquid, or applying suction or negative pressure, to handle 20 and/or insertion portion 30. For example, the gas or the liquid may be applied, or delivered, through one or more channels, or lumens, of medical device 15.

Handle 20 may include one or more actuators, for example, a first actuator 22 and/or a second actuator 24. First actuator 22 and/or second actuator 24 may include, for example, rotatable knobs that rotate to push/pull one or more elements (e.g., articulation wires or cables) that extend through one or more portions of insertion portion 30 and connect to distal end portion 40 of insertion portion 30. For example, first actuator 22 and/or second actuator 24 may be configured to rotate about a respective axis to push/pull actuating elements that extend within one or more lumens of insertion portion 30. Rotation of first actuator 22 and/or second actuator 24 may cause a portion of insertion portion 30 (e.g., distal end portion 40) to bend, for example, via an articulating joint (not shown). Additionally or alternatively, handle 20 may include one or more additional actuators (e.g., buttons, knobs, levers, locks, etc.) to, for example, limit movement of first actuator 22 and/or second actuator 24, close or open an end effector, rotate an end effector about a longitudinal axis, raise or lower an elevator to move a device delivered through the working channel, capture an image and/or video, and/or provide other functionality to an end effector and/or distal tip 50.

Handle 20 may also include one or more valves, for example, a first valve 26 and a second valve 28. Although two valves (i.e., first valve 26 and second valve 28) are shown, handle 20 may include additional (e.g., a third valve, a fourth valve, etc.) or fewer valves (e.g., no valves or only first valve 26). In some embodiments, first valve 26 may be configured to control the supply of air and/or water to distal tip 50. Second valve 28 may be configured to control the application of suction to distal tip 50. Additional valves may be used, for example, to control the application of one or more medicines, agents, materials, etc. from distal tip 50.

Handle 20 may also include a port or proximal opening 29, which may be fluidly connected to one or more lumens and/or a working channel of insertion portion 30. For example, a medical instrument (not shown) may be inserted into proximal opening 29 and may be extended to and/or distally from distal tip 50 via the one or more lumens. The lumen(s) may have a distal opening 70 on distal face 50D of distal tip 50, which may be a distalmost end of distal end portion 40. Distal opening 70 may be fluidly coupled to proximal opening 29 of handle 20. In such a way, a medical instrument inserted into proximal opening 29 may be extended distally to distal opening 70. In some aspects, the medical instrument may be extended distally from distal opening 70, for example, distal of distal tip 50. Additionally or alternatively, one or more materials (e.g., liquids, gels, gasses, patches, powders, etc.) may be supplied to a target site via distal opening 70. Additionally or alternatively, suction may be applied to the target site from distal opening 70 (e.g., via proximal opening 29 or via umbilicus 60), for example, to remove fluid and/or debris from the target site.

One or more portions of insertion portion 30 may be flexible and formed of medical grade material suitable for accessing a tortuous path within the body. The rigidity/flexibility of insertion portion 30 is not limited. In some embodiments, the rigidity/flexibility of insertion portion 30 may vary, for example, along a longitudinal length of insertion portion 30. A longitudinal length of insertion portion 30 may vary and is not limited. For example, insertion portion 30 may have a length of approximately 5-100 inches, for example, between 10-70 inches. Furthermore, an outer diameter of insertion portion 30 is not limited. For example, the outer diameter of insertion portion 30 may be approximately 0.07 to 0.60 inches, for example, between 0.10-0.50 inches. In some embodiments, the outer diameter of insertion portion 30 may vary along the longitudinal length. For example, the outer diameter of a proximal portion of insertion portion 30 may be less than or greater than the outer diameter of distal end portion 40 of insertion portion 30, or vice versa.

A cross-section of insertion portion 30 and/or distal tip 50 may be generally circular, ovular, or any other shape commonly used in the art for the insertion portion of a medical device. As mentioned above, although not shown, distal end portion 40 may include one or more articulation joints configured to articulate distal tip 50 in one or more directions (e.g., left, right, up, down, etc.). Although not shown, distal end portion 40 may further include one or more treatment or accessory devices (e.g., laser fibers, elevators, etc.), and/or one or more other devices to otherwise image, view, or otherwise treat a target site.

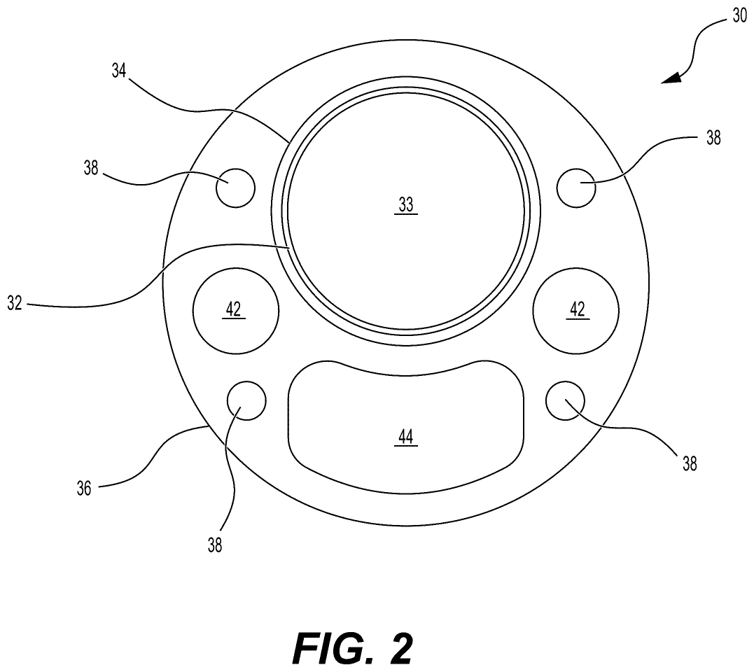

FIG. 2 is a cross-sectional view of insertion portion 30 at any point along its longitudinal axis. Insertion portion 30 also may be called a medical device shaft, and this disclosure will use the same reference numeral 30 to refer to the shaft.

In embodiments, shaft 30 includes three components. A first component may be a tubular sheath or liner 32 having an inner diameter of approximately 0.05 to 0.25 inches (and in other embodiments approximately 0.06 to 0.09 inches) and a thickness of approximately 0.0005 to 0.0100 inches. A single lumen inside of liner 32 may define a working channel 33 for shaft 30, receiving accessory tools therethrough (such as forceps, knives, graspers, needles, baskets, laser lithotripters, etc.).

Liner 32 may be manufactured by co-extruding two layers of materials over a core mandrel. A first, inner layer of liner 32 may define an internal surface of working channel 33. The inner layer of liner 32 be made of a resin, such as a high molecular weight polyethylene, that provides lubricity to working channel 33.

In embodiments, the resin of liner 32 may have a molecular weight of approximately 200 kilodaltons to over 1 megadalton (and in other embodiments approximately 200 to 500 kilodaltons). Resins having a higher molecular weight (e.g., approximately more than 200 kilodaltons to less than 1 megadalton) may have a higher viscosity and may create lubricious surfaces. For example, as the molecular weight of the resin increases, the inner layer of liner 32 may become more lubricious (e.g., a coefficient of friction of the inner layer may decrease). Liner 32 may be manufactured by conventional melt thermoplastic processes. The conventional melt thermoplastic processes may include injection molding, blow molding, extrusion, thermoforming, rotational molding, and/or any other process for processing thermoplastics.

The inner layer of liner 32 may alternatively be comprised of resin having an ultrahigh molecular weight (e.g., approximately more than 1 megadalton). In such examples, the resin having an ultrahigh molecular weight may be processed by film wrapping and/or other solid state processing technologies. The solid state processing technologies may include, but are not limited to, solid state coextrusion, powder extrusion, compression molding, gel spinning, and/or sintering.

In other aspects, the inner layer of liner 32 may be comprised of alternative resins and/or materials. For example, the inner layer of liner 32 may be comprised of polytetrafluoroethylene (PTFE) and/or additive-filled polymers. As such, the PTFE or additive-filled polymers may also provide lubricity to working channel 33.

A second, outer layer of liner 32 may be made of an acid-modified polyethylene that will function as a tie layer (e.g., assisting with the adhesion of the inner layer of liner 32 to a braid 34 and/or to a multi-lumen extrusion (MLE) 36, as will be described). Acid-modified polyethylene may be used, for example, due to the material's compatibility with the high molecular weight polyethylene comprising inner layer of liner 32. In some examples, the tie layer may include an acid or anhydride modifier (e.g., an epoxide, a silane, a maleic acid, an anhydride, or other carboxylic acid, etc.), for example, to improve compatibility with the inner layer of liner 32. The tie layer may also be a copolymer of polyethylene.

The outer layer of liner 32 may be made from other materials that are compatible with the inner layer of liner 32. For example, if inner layer of liner 32 is comprised of a PTFE or a PFA, a modified PTFE or a modified PFA may be used, for example, to improve compatibility between each layer of liner 32. In other embodiments, liner 32 may include only one layer, for example, omitting the outer tie layer. In further embodiments, liner 32 may be comprised of additional layers, for example, three layers, four layers, etc. Additional layers may be used, for example, to assist in manufacturing liner 32 where two or more layers are incompatible with each other. For example, a middle layer may be used between an outer layer and an inner layer of liner 32 that are incompatible with each other. The middle layer may be used to adhere the two incompatible layers.

A second component of the shaft is a braid 34. Braid 34 may have an outer diameter of approximately 0.07 to 0.30 inches (and in other embodiments approximately 0.07 to 0.08 inches) and a thickness of approximately 0.001 to 0.010 inches. Braid 34 overlays liner 32. For example, braid 34 may surround and contact liner 32. Braid 34 may overlay an entire longitudinal length of liner 32. Alternatively, braid 34 may overlay one or more discrete lengths of liner 32. In some examples, braid 34 may be or resemble a braided tube. In other examples, braid 34 may be a coil or a spring.

Braid 34 may be manufactured over liner 32 via any suitable process, including while liner 32 remains on the core mandrel. Manufacturing braid 34 directly over liner 32 may decrease manufacturing costs, for example, due to the reduction of secondary, or post-processing, processes that may be required for separately manufacturing and applying braid 34 to liner 32. Alternatively, braid 34 may be manufactured separately from liner 32 and applied to liner 32 in a separate, or secondary, manufacturing process. Separately manufacturing braid 34 from liner 32 may be beneficial, for example, in embodiments where braid 34 is applied to one or more discrete lengths of liner 32.

The outer layer of liner 32 (i.e., the tie layer) may assist in adhering braid 34 to the inner polyethylene layer of liner 32. For example, in embodiments, some of the outer layer of liner 32 may bleed into or through braid 34 to assist in adhering liner 32 to braid 34. Braid 34 may be made of any suitable number and shape of wires of any suitable material. For example, braid 34 may be wound or woven in any suitable pattern, angle, pitch, and density, to achieve the desired flexibility, column strength, and other characteristics of shaft 30 and working channel 33. For example, the wires may be round or flat, metal (e.g., stainless steel) wires wound in a two-over-two pattern at approximately 70 pic/inch. In embodiments, little to no spacing exists between adjacent windings of braid 34. No space between the windings results in a smoother working channel 33 within liner 32, thus enhancing passability of tools through working channel 33.

A third component of shaft 30 is a multi-lumen extrusion (MLE) 36, having an outer diameter of approximately 0.05-0.60 inches (and in other embodiments approximately 0.13 to 0.16 inches). MLE 36 may define multiple lumens. For example, MLE 36 may include seven lumens in addition to working channel 33. The seven additional lumens may include four relatively small diameter lumens 38 for receiving articulation, or steering, wires (e.g., permitting articulation in left, right, up, and down directions), two relatively medium size lumens 42 for irrigation and insufflation, and one relatively large lumen 44 to receive the wires or other circuitry used for imaging and lighting. Additional or fewer lumens can be provided in MLE 36, and the lumens can have any suitable cross-sectional shape, including circular, ovular, crescent-shaped, square, etc.

MLE 36 may be made of a melt thermoplastic material, such as polyesters, polyethylenes, PEEK, polyamides (nylons), and any other melt thermoplastic capable of being processed via conventional melt extrusion technologies. The specific melt thermoplastic material used with MLE 36 may be based on the desired flexibility and/or other properties of the resulting shaft 30. In aspects, it may desirable to form MLE 36 using melt thermoplastic materials, for example, over materials having a high molecular weight. In particular, the thin walls of multilumen extrusions may be more easily manufactured using melt thermoplastic materials.

Furthermore, by forming MLE 36 from a melt thermoplastic material and liner 32 from a resin with a high molecular weight, the properties of MLE 36 and liner 32 may differ and may be tailored to a desired application. For example, MLE 36 may be more flexible and/or tackier, as compared to liner 32. In other aspects, liner 32 may be more flexible than MLE 36. Liner 32 may be more lubricious and/or have a greater radial strength than MLE 36.

MLE 36 may be extruded over braid 34 (and liner 32) via any suitable method, for example, including extruding MLE 36 over braid 34 while braid 34 and liner 32 remain on the core mandrel. Accordingly, MLE 36 may form an outer tube that surrounds and at least partially contacts braid 34 and/or liner 32. In other aspects, MLE 36 may be separately formed (e.g., not extruded over braid 34 or liner 32). Once MLE 36 has been extruded, braid 34 (and liner 32) may then be inserted through the appropriate lumen of MLE 36 such that MLE 36 forms an outer tube that surrounds and at least partially contacts braid 34 and/or liner 32.

In some aspects, components comprised of melt thermoplastic materials may be tacky, or sticky. Thus, if an entire working channel of a medical device were to be comprised of a melt thermoplastic material, inserting or removing accessory devices via the working channel may be difficult. However, with liner 32 forming working channel 33 (as opposed to the walls of MLE 36 forming working channel 33), accessory devices may be inserted or removed more easily. For example, as previously discussed, liner 32 may be formed from lubricious materials, thus permitting easy insertion or removal of accessory devices. In some aspects, liner 32 may serve as a barrier between working channel 33 and MLE 36.

The additional lumens of MLE 36 (e.g., lumens 38, 42, 44) may be created via any suitable method, such as using additional core mandrels to form each lumen and/or using air pressure to form each lumen. Working channel 33 and the additional lumens of MLE 36 may be formed within a same extrusion head. For example, air pressure may be used to form working channel 33 and tube-free extrusion processes may be used to form the additional lumens (e.g., lumens 38, 42, 44). In embodiments, some of the tie layer of liner 32 may bleed through braid 34 to assist in adhering the thermoplastic MLE 36 to braid 34.

Each of the parts of shaft 30 (e.g., liner 32, braid 34, and MLE 36) can extend the entire length of shaft 30 from the medical device handle 20 to distal tip 50 (for example, to a distal end cap). Having these three parts extend the entire shaft length eases manufacture (e.g., due to the limited number of parts). Furthermore, because the parts of shaft 30 (e.g., liner 32, braid 34, and MLE 36) may be extruded or formed together, shaft 30 may be spooled. A spool-type construction may decrease cost of shaft 30.

MLE 36 may also form an articulation portion of shaft 30 (e.g., distal end portion 40). For example, a distal portion of MLE 36 may include one or more notches or cut outs, for example, to permit bending of MLE 36. In embodiments, the articulation portion of shaft 30 may include a separate distal MLE. The distal MLE may be coupled to a distalmost end of MLE 36. For example, the distal MLE may have any or all of the same physical characteristics of MLE 36. For example, the distal MLE may have the same dimensions and shape as MLE 36 and/or the distal MLE may have the same number, shape, and/or size of the lumens extending therethrough. The distal MLE may be coupled to MLE 36 such that the lumens of MLE 36 are aligned and in fluid communication with the lumens of distal MLE. For example, liner 32 and braid 34 may extend from MLE 36 and into, or through, a working channel of the distal MLE. As such, MLE 36 and the distal MLE may form a continuous shaft 30.

Distal MLE may be formed using any material or any method previously described for forming MLE 36. Distal MLE may be more flexible, or softer, than MLE 36. Because distal MLE is comprised of a more flexible, or softer material, distal MLE may articulate more easily. In some aspects, distal MLE may further include one or more notches or cutouts, for example, to facilitate articulation of the distal MLE.

Distal MLE may be coupled to MLE 36 via any number of processes. For example, approximately 0.10 to 4.00 inches (and in other embodiments approximately 1.0 to 2.0 inches) of a distal portion of MLE 36 may be removed. Liner 32 and braid 34 may remain untouched. As such, with the distal portion of MLE 36 removed, liner 32 and braid 34 may extend past a distal end of MLE 36. Such a configuration may also be accomplished by extending liner 32 and braid 34 distally past a distalmost end of MLE 36 (thereby eliminating the need to remove a distal portion of MLE 36). The distal MLE may then slide over liner 32 and braid 34 such that a proximal end of the distal MLE abuts a distal end of MLE 36. In some examples, MLE 36 and the distal MLE may then be fixed together by, for example, using an adhesive, a swage or crimp, or any other means for connecting two tubes. Additionally or alternatively, distal MLE may be fixed to liner 32 and/or braid 34 by, for example, using an adhesive, a swage or crimp, or any other means for connecting two tubes.

In aspects, shaft 30 can consist essentially of liner 32, braid 34, and MLE 36, without any other layers or tubes. In other aspects, shaft 30 may further comprise one or more outermost layers (e.g., surrounding MLE 36). The outermost layer(s) may provide structural support to shaft 30. For example, the outermost layer(s) may include one or more of a braid, a coil, or an additional tubular member. In embodiments, the outermost layer(s) may vary along a longitudinal length of shaft 30, for example, thereby forming a shaft having variable stiffness. For example, the material comprising the outermost layer(s) may vary and/or a thickness of the outermost layer(s) may vary. In some aspects, the outermost layer(s) may provide a barrier between the subject and MLE 36. The outermost layer(s) may also be lubricated or include one or more additives to increase the lubricity of shaft 30. In these aspects, the lubrication or additives of outermost layer(s) may provide for easy insertion of shaft 30 into the subject.

Having braid 34 surrounding only liner 32 and thereby only working channel 33 of shaft 30 (as opposed to surrounding other lumens or walls of MLE 36) reinforces the portions of the medical device shaft 30 that typically need the greatest reinforcement. Such a configuration may focus the protection provided by braid 34 on working channel 33. For example, braid 34 may inhibit an accessory instrument from perforating through the walls of liner 32 that define working channel 33. This may be especially important when shaft 30 is navigating tight bends, and the distal end of the accessory instrument is relatively sharp, for example, a needle. Braid 34 may also inhibit working channel 33 from buckling and potentially closing at tight bends.

Such a configuration may also allow for lumens 38, 42, 44 of MLE 36 to expand and contract without being constrained by the braid. With lumens 38, 42, 44 more freely expanding and contracting, buckling of the inner webs (e.g., the walls of MLE 36 between each lumen) may be reduced. As such, the shape of lumens 38, 42, 44 and/or MLE 36 may be maintained. Additionally or alternatively, a torsional moment of shaft 30 may improve with braid 34 surrounding liner 32. For example, because the reinforcement of braid 34 is more central to shaft 30 (as opposed to surrounding MLE 36), the torsional moment of shaft 30 may be improved, for example, to allow for better torque transfer down a longitudinal length of shaft 30.

In addition, as opposed to shafts that may include a braid surrounding an entirety of insertion portion 30 (e.g., as an outermost layer of MLE 36), embodiments described herein may use lesser amounts of wires, decreasing cost.

In other embodiments, additional braids, or coils, may be provided in one or more other lumens of MLE 36 (e.g., lumens 38, 42, 44). The additional braids may be similarly constructed to that of braid 34. In some embodiments, the additional braids may provide additional structural support within lumens 38, 42, 44 and/or to MLE 36. In some aspects, the additional braids may surround one or more components extending through each respective lumen. For example, the additional braids may surround one or more articulation wires, camera wires, fluidics tubes, etc. The additional braids may extend through a proximal portion of MLE 36, for example, terminating just before the articulation portion of MLE 36 (or the distal MLE fixed to the distal end of MLE 36). As such, the proximal portion of MLE 36 may be stiffer as compared the distal portion of MLE 36 (or the distal MLE).

Though various dimensions and materials of parts are provided in this disclosure, embodiments are not limited to any specific size or material. It will be apparent to those skilled in the art that various modifications and variations can be made to the disclosed device without departing from the scope of the disclosure. For example, it will be apparent to those skilled in the art that various aspects of each configuration discussed herein may be used with other configurations.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical device shaft, comprising:
a tubular liner defining a working channel capable of receiving an accessory instrument;
a braided tube surrounding and contacting the tubular liner; and
an outer tube surrounding and contacting the braided tube.

2. The medical device shaft of claim 1, wherein the tubular liner includes an inner layer and an outer layer surrounding the inner layer.

3. The medical device shaft of claim 2, wherein the inner layer comprises polyethylene, the outer layer comprises an acid-modified polyethylene, and the inner layer is co-extruded with the outer layer.

4. The medical device shaft of claim 3, wherein the polyethylene of the inner layer has a molecular weight of at least 200 kilodaltons.

5. The medical device shaft of claim 3, wherein the outer layer adheres the braided tube to the inner layer.

6. The medical device shaft of claim 1, wherein the tubular liner includes only one layer comprising an inner layer, and wherein the inner layer comprises polyethylene having a molecular weight of at least 200 kilodaltons.

7. The medical device shaft of claim 1, wherein the braided tube surrounds only one lumen, the only one lumen comprising the working channel.

8. The medical device shaft of claim 1, wherein the braided tube includes metal wires, and adjacent windings of the braided tube contact one another.

9. The medical device shaft of claim 1, wherein the outer tube is an extruded thermoplastic defining multiple lumens.

10. The medical device shaft of claim 9, wherein the multiple lumens include at least one steering lumen for accommodating a steering wire, at least one irrigation lumen capable of providing irrigation fluid, and at least one electronics lumen for accommodating electrical circuitry.

11. The medical device shaft of claim 10, wherein each of the at least one steering lumen, the at least one irrigation lumen, and the at least one electronics lumen is surrounded by only the outer tube.

12. The medical device shaft of claim 1, consisting essentially of the tubular liner, the braided tube, and the outer tube.

13. The medical device shaft of claim 1, wherein the tubular liner is extruded on a mandrel to form the working channel, the braided tube is formed onto the tubular liner while the tubular liner is on the mandrel, and the outer tube is extruded onto the braided tube while the tubular liner is on the mandrel.

14. A medical device having a medical device shaft of claim 1, the medical device further comprising:
a handle directly attached to a proximal end of the medical device shaft; and
an end cap directly attached to a distal end of the medical device shaft.

15. The medical device of claim 14, wherein the handle includes steering components, and a port in communication with the working channel, and the end cap includes an imager, lighting, and an exit port in communication with the working channel.

16. A medical device shaft, comprising:
a tubular liner including a polyethylene layer and a tie layer surrounding the polyethylene layer, wherein the polyethylene layer defines a working channel capable of receiving an accessory instrument;
a braided tube of metal wires surrounding and contacting the tie layer, wherein the tie layer enhances adhesion of the braided tube to the polyethylene layer, and wherein the braided tube surrounds only one lumen comprising the working channel; and
a first thermoplastic outer tube surrounding and contacting the braided tube, wherein the first outer tube defines a first plurality of lumens therethrough that are surrounded by only the first outer tube.

17. The medical device shaft of claim 16, further comprising a second thermoplastic outer tube surrounding and contacting the braided tube, wherein the second thermoplastic tube is distal to the first thermoplastic tube, wherein the second outer tube defines a second plurality of lumens therethrough that are surrounded by only the second outer tube, and wherein the second outer tube has a stiffness that is less than a stiffness of the first outer tube.

18. A method of making a medical device shaft, the method comprising:
extruding a tubular liner onto a mandrel;
forming a braided tube onto the tubular liner while the tubular liner is on the mandrel; and
after the forming step, extruding an outer tube onto the braided tube while the tubular liner is on the mandrel.

19. The method of claim 18, wherein extruding the tubular liner onto the mandrel includes co-extruding an inner polyethylene layer and a tie layer surrounding the inner polyethylene layer.

20. The method of claim 18, wherein the outer tube includes a thermoplastic and defines multiple lumens surrounded by only the outer tube, the multiple lumens including at least one steering lumen for accommodating a steering wire, at least one irrigation lumen capable of providing irrigation fluid, and at least one electronics lumen for accommodating electrical circuitry.
